(19) United States
(12) Patent Application Publication
(10) Pub. No.: US 2026/0354817 A1
(43) Pub. Date: Nov. 12, 2026

(54) Suture with Stellate Core for Surgical Use

(71) Applicant: Cannuflow, Inc., Scotts Valley, CA (US)

(72) Inventor: Theodore R. Kucklick, Scotts Valley, CA (US)

(73) Assignee: Cannuflow, Inc., Scotts Valley, CA (US)

(21) Appl. No.: 19/649,821

(22) Filed: Apr. 16, 2026

Related U.S. Application Data
(60) Provisional application No. 63/791,143, filed on Apr. 18, 2025.
(60) Provisional application No. 63/836,994, filed on Jul. 1, 2025.
(60) Provisional application No. 63/990,756, filed on Feb. 25, 2026.

(51) Int. Cl. A61B 17/06

(52) U.S. Cl. CPC A61B 17/06166; A61B 2017/06176

(57) ABSTRACT

A stellate or star-shaped cross-section sutures that include collagen containing coatings, including solid and hollow core sutures and associated methods of manufacturing such sutures. Methods for coating the surgical sutures and implantable medical filaments for use as surgical sutures and implantable medical filaments. The stellate or star shaped suture increases surface area and coating retention for sutures.

### CROSS-REFERENCE TO RELATED APPLICATIONS

This application claims the benefit of priority to U.S. Provisional Application 63/791,143, filed Apr. 18, 2025, 63/836,994, filed Jul. 1, 2025, and 63/990,756, filed Feb. 25, 2026.

### FIELD OF THE INVENTIONS

The inventions described below relate to the field of surgical sutures and implantable medical filaments. More specifically, stellate or star-shaped cross-section sutures that include collagen containing coatings, including solid and hollow core sutures and associated methods of manufacturing such sutures. The inventions also describe methods for coating the surgical sutures and implantable medical filaments.

### BACKGROUND OF THE INVENTIONS

Surgical sutures are widely used in wound closure and tissue repair. Conventional sutures are typically formed from monofilament or braided fibers having circular cross-sections. While such sutures provide adequate mechanical strength, they often exhibit limitations in promoting tissue integration and delivering biologically active materials.

Coated sutures, including those incorporating collagen or other bioactive materials, have been developed to enhance tissue healing and reduce inflammation. However, conventional cylindrical fibers provide limited surface area for coating retention and may not effectively localize or retain bioactive materials.

In addition, emerging surgical techniques increasingly require sutures capable of performing additional functions, such as sensing, signal transmission, or localized delivery of therapeutic agents. Existing suture designs do not readily accommodate such functionalities.

There is a need for improved suture designs that provide enhanced surface area for coating retention, improved mechanical and biological performance, and compatibility with advanced manufacturing techniques and integrated functional components.

### SUMMARY

The devices and methods described below provide for a collagen coated suture with a core that includes at least one or a plurality of star or stellate shaped fibers. The inner structure of the collagen core suture includes a central bundle made of a plurality of collagen coated stellate fiber bundles or filaments. Alternatively, the central core can be a single stellate shaped fiber. Each fiber has a star shaped or stellate (multi point) cross section geometry rather than being solid round or cylindrical. The points or arms of the star radiate from a central core to create channels or grooves between them. The stellate shape of the fiber bundle or filament maximizes the amount of collagen that is on the filament. Collagen is coated over the core so that the collagen can be either crosslinked or uncrosslinked but does not need to be structural. The star shaped fiber or filament architecture can optimize for structure, or absorbability, or bioinduction, and offers the ability to blend these parameters in one platform to offer an optimized solution for every surgical indication. The stellate cross-section fiber offers improved tensile strength over time, as well as greater simplicity and potentially lower cost of manufacture than the prior art.

The collagen coated suture can optionally include at least one or a plurality of star or stellate shaped fibers wither the fibers include a hollow core defines by a central lumen. The central lumen extends longitudinally along the entire length of the suture. The inner structure of the collagen core suture includes a central bundle made of a plurality of collagen coated stellate fiber bundles or filaments. Alternatively, the central core can be a single stellate shaped fiber. Each fiber has a star shaped or stellate (multi point) cross section geometry rather than being solid round or cylindrical and has a hollow core. The center of the fiber is hollow, formed of a central lumen through the entire fiber. The points or arms of the star radiate from a central core to create channels or grooves between them. Collagen is coated over the core so that the collagen can be either crosslinked or uncrosslinked but does not need to be structural.

### BRIEF DESCRIPTION OF THE DRAWINGS

FIG. 1a illustrates a composite filament for a surgical suture having a single stellate shaped fiber forming the core and an outer overbraid over the core.

FIG. 1b illustrates a cross section of the suture of FIG. 1a.

FIG. 2a illustrates a composite filament for a surgical suture having a plurality of stellate shaped fibers forming the core and an outer overbraid over the core.

FIG. 2b illustrates a cross section of the suture of FIG. 2a.

FIG. 3 illustrates an individual fiber of the core or central bundle of FIGS. 1a-2b.

FIGS. 4a-4c illustrate a composite filament for a surgical suture having a plurality of the composite filaments with stellate shaped fibers, non-overbraided outer covering.

FIGS. 5a and 5b illustrate a composite filament for a surgical suture that includes a stellate shaped fiber with a hollow core.

FIG. 5c is a cross-sectional view of the composite filament for a surgical suture of FIGS. 5a and 5b.

FIG. 6 illustrates a composite filament for a surgical suture that includes a plurality of stellate shaped fibers with a hollow core.

FIG. 7 illustrates an alternative stellate core suture with barbs 12

FIGS. 8 and 9 illustrate a method of manufacturing a stellate surgical suture fiber.

FIGS. 10a and 10b Figures illustrate another method of manufacturing a stellate core fiber using a solution-based coating process.

### DETAILED DESCRIPTION OF THE INVENTIONS

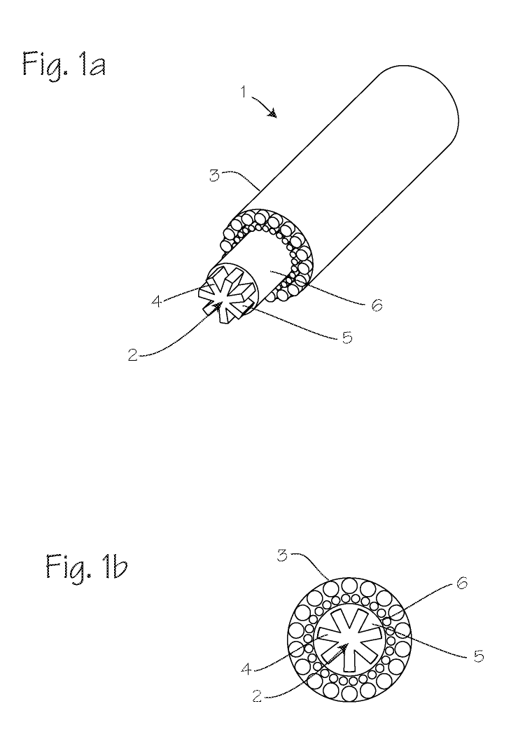

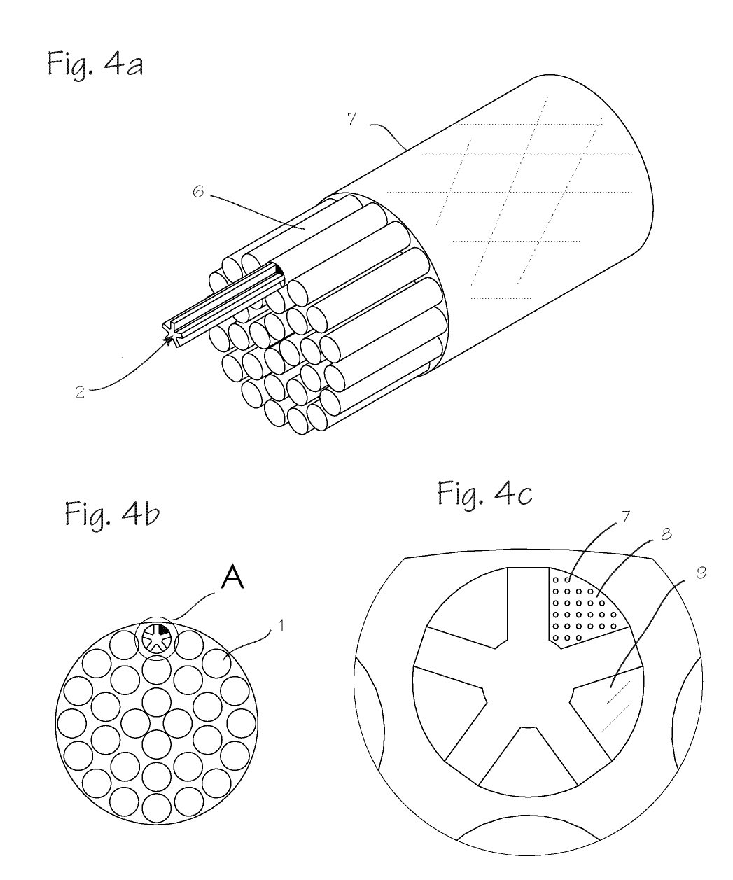

FIG. 1a illustrates a composite filament 1 for a surgical suture having a single stellate shaped fiber 2 forming the core and an outer overbraid sheath 3 that surrounds and consolidates the core. The core includes a stellate cross-sectional geometry defined by a plurality of radially extending arms 4 separated by longitudinal channels 5 extending along a length of the fiber. The single stellate shaped fiber has a multi-point cross section geometry, shown in FIG. 1b, rather than being solid round or cylindrical. The points or arms of the star radiate from a central core to create channels or grooves between them. The stellate core is also coated by a collagen layer 6. The stellate cross-section fiber is preferably not absorbable. The collagen coating is disposed at least partially within the longitudinal channels defined by the stellate geometry in order to maximize retention of the coating along the length of the fiber. The coating includes a composite matrix that includes collagen with micronized particles and a polymer binder. The features of the coating are illustrated in detail in FIG. 4c. The collagen particles have a size in the range of approximately 1 micron to 5 microns. The collagen in the coating is in an amount between approximately 5% and 80% by volume. The polymer binder can include polycaprolactone (PCL) that is dissolved in a solvent such as dichloromethane (DCM), chloroform, or hexafluoro-2-propanol (HFIP) to form a coating solution prior to application. The coating solution can be applied to the elongate fibers and dried to form a coating layer retained within the stellate channels. The fiber can be made of a high-tensile strength non-absorbable polyethylene or non-absorbable polyester fiber. The outer overbraid is made of an ultra-high molecular weight polyethylene (UHMWPE) or a polyester overbraid in a single or double layer. The individual star shaped fibers increase the surface area significantly to allow more collagen to be carried by the filament than by only coating a round surface. The use of a non-absorbable fiber can be advantageous when optimizing for maximum tensile strength over time while retaining the advantage of a maximized volume of bioinductive collagen for tissue ingrowth that the collagen coating onto the stellate cross section provides. This is advantageous in high-load orthopedic repairs where mechanical structure is required. The arms of the stellate core also direct tendon fiber growth along its length that can mimic native tendon. The advantage is this construct should have good tensile strength and be very flexible. It will also direct cell growth along the grooves and channels.

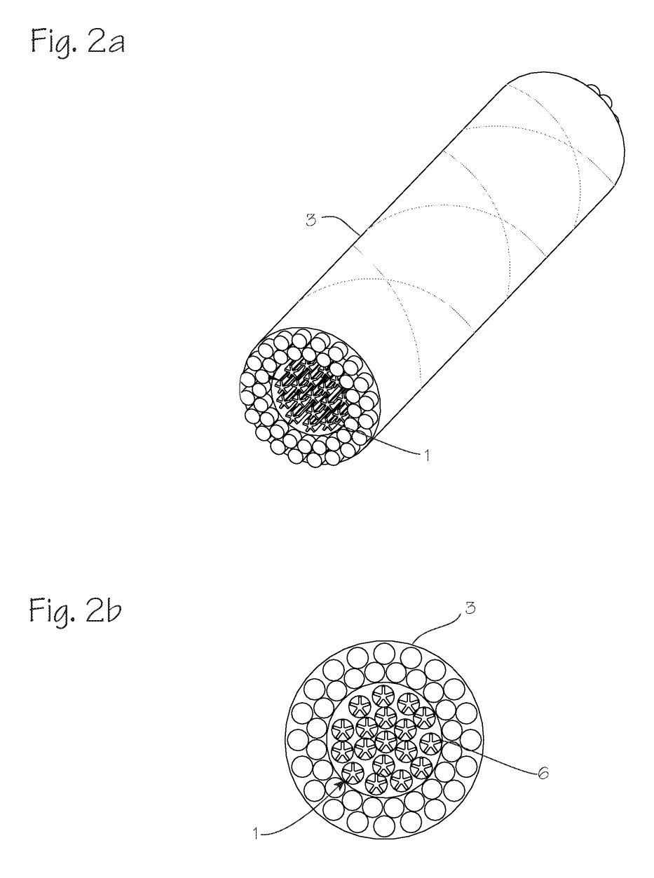

FIG. 2a illustrates a composite filament for a surgical suture having a plurality of the composite filaments 1 with stellate shaped fibers 2 forming the core and an outer overbraid 3 over the multi fiber core. Each individual fiber is star shaped or stellate, having a multi-point cross section geometry. The stellate cross-section fibers are individually collagen coated 3 and then bundled into the suture core. The plurality of fibers forms the core or central bundle. The central bundle is formed of a plurality of stellate shaped fibers that may be braided to form the central core. The central core is collagen coated 6 and contained by an outer overbraid 3.

The fibers can be made of a high-tensile strength non-absorbable polyethylene or non-absorbable polyester fiber. The core can also be a silicone thermoset material with a star or stellate shape. The core construction can be molded with a stiff aglet end for suture threading and for insertion into a swaged needle. Each fiber is a collagen coated stellate multi fiber bundle.

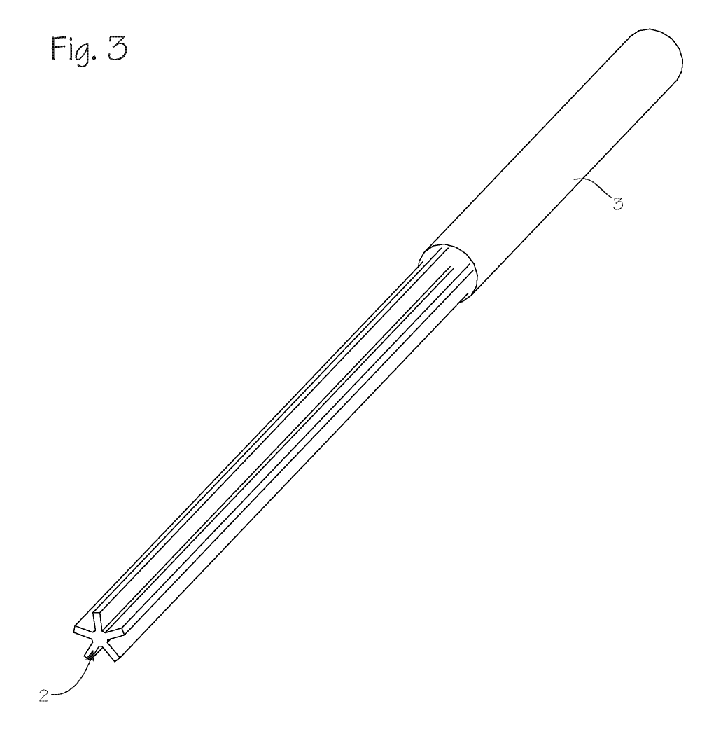

FIG. 3 illustrates an individual fiber of the core or central bundle of FIGS. 1a-2b. Each fiber includes a 15 to 250 micron diameter stellate shaped, absorbable polymer fiber 2. Each individual fiber is coated with collagen 6. The stellate fibers can be made of any absorbable polymer for example, polydioxanone (PDO), polylactic acid (PLA), polyglycolic acid (PGA) or PUUR (polyurethane-urea) and PUUR-PCL alloys.

FIGS. 4a-4c illustrate a composite filament 1 for a surgical suture having a plurality of the composite filaments with stellate shaped fibers 2 in a non-overbraided outer covering 7. The non-overbraided outer covering is a monofilament or uniformly braided structure. The non-overbraided suture lacks a distinct outer sheath surrounding a separate core but includes the inner stellate shaped fibers as described above. The stellate shaped fibers include a plurality of fibers that are interwoven with one another without forming a discrete internal core and outer sheath. The filament includes between 20 and 30 braided strands. The filament could include more or less strands. Each of the stellate shaped fibers is structurally equivalent and participate uniformly in load bearing. FIG. 4b illustrates a cross-sectional view of the composite filament of FIG. 4a. It shows the individual braid strands. Each strand is collagen coated stellate fiber. The individual strands shown are 20 to 150 microns and form a yarn bundle for a USP suture size between 5-0 and 1 (150-500 microns). FIG. 4c illustrates the detail of Section A of FIG. 4b. The longitudinal channels of each strand are filled with micronized collagen particles 7 having a size between 1 and 5 microns in size and 5% by 80% filler load by volume and a binder 8. The micronized collagen and polymer create a matrix 9 within the longitudinal channels of the fiber.

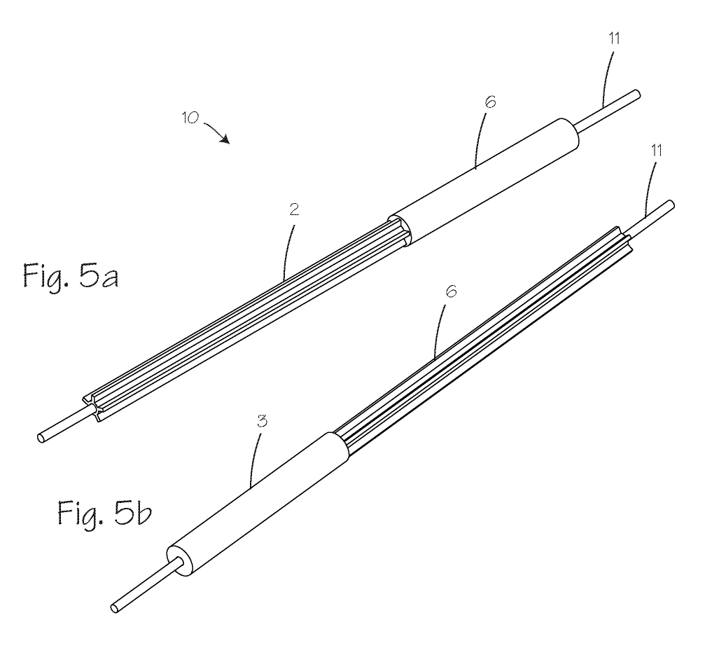

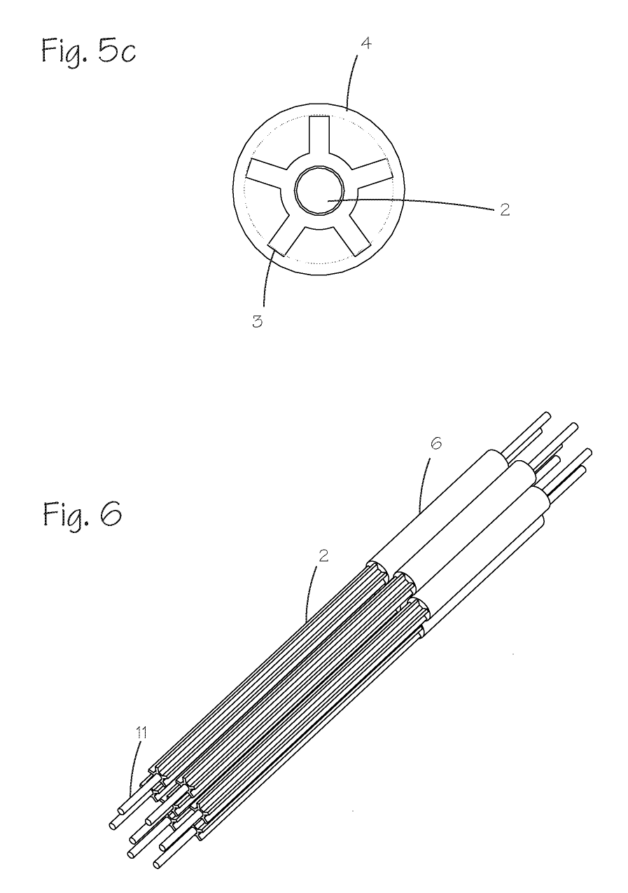

FIGS. 5a and 5b illustrate a composite filament 10 for a surgical suture that includes a stellate shaped fiber 2 with a hollow core 11 (shown in FIG. 5c) that forms a central lumen that extends longitudinally along the entire length of the surgical suture. The suture includes a stellate cross-section with multiple longitudinal points or arms 4 that project from the outer surface of the central lumen. The composite filament includes a collagen coating 6 at the distal end of the filament shown in FIG. 5a. FIG. 5b illustrates the collagen coating at the proximal end of the composite filament. This figure shows the core and coating stripped back for clarity to show its layered construction. The points or arms of the star radiate from the central core to create channels or grooves between them. The stellate fiber when coated forms an outer surface of the suture. The coating is contained within the channels or grooves created by the points or arms. The central lumen can be used for several purposes. A signal wire 11 can be contained within the central lumen and extend through the distal end of the central lumen. The proximal end of the signal wire can be operatively connected to a sensor. The central lumen protects and isolates the signal wire, enabling real-time pressure or temperature sensing at or near a surgical site. The suture can be connected to a signal generator at one end and an energy application electrode at the other end, or a sensor and a signal processor, or a combination of power and sensor in a closed-loop control system. The outer surface may also be coated with a biocompatible material to improve insertion. The central lumen can alternatively house electronic leads for delivering stimulation or for sensing. Alternatively, the central lumen can also serve to flow bioactive material filing or other fluid delivery. The central lumen may be extruded through the stellate body or formed by molding or fiber braiding with a hollow core. The central lumen can achieve directional or localized fluid delivery. The suture can have a wire co-extruded into the suture for electrical signals or energy delivery. For example, a soft wire without tensile strength, such as silver, can add tensile strength by co-extruding a jacket of ultra high molecular weight polyethylene (UHMWPE). The center lumen can be hollow for pressure sensing, fluid delivery, or fluid removal by wicking. FIG. 5c is a cross-sectional view of the suture of FIGS. 5a and 5b. The stellate fiber includes multiple points or arms that project to form a star shape. The fibers have a diameter in the range of 10 to 700 microns (#11-0 to #3 suture USP size). The central lumen can have a diameter in the range of 5 to 100 microns. The stellate core can be coated with collagen, antibiotics, anti inflammatories or biologic adjuvants. FIG. 6 illustrates a composite filament 10 for a surgical suture that includes a plurality of stellate shaped fibers with a hollow core.

The signal wires may be comprised of materials such as silver, platinum, MP35N, cobalt-chrome or Nitinol. The signal wire can be conductive or optical element operably connected to external monitoring equipment for live time data transmission. It can be connected to a pressure sensor located at the distal end of the lumen to detect pressure. The signal wire is disposed within the central lumen to allow sensing of pressure, tension or strain at a surgical repair site. The signal wire may be micro-gauge insulated conductive wire. Alternatively it can be an optical fiber for transmitting light based signals. Optionally, it can also be flexible printed circuit (FPC).

The stellate fiber materials can be comprised of UHMWPE for tensile strength. Alternatively, the fiber materials can be made of any absorbable polymer for example polydioxanone (PDO), polylactic acid (PLLA) or polyglycolic acid (PGA), polydioxanone, or polycaprolactone (PCL).

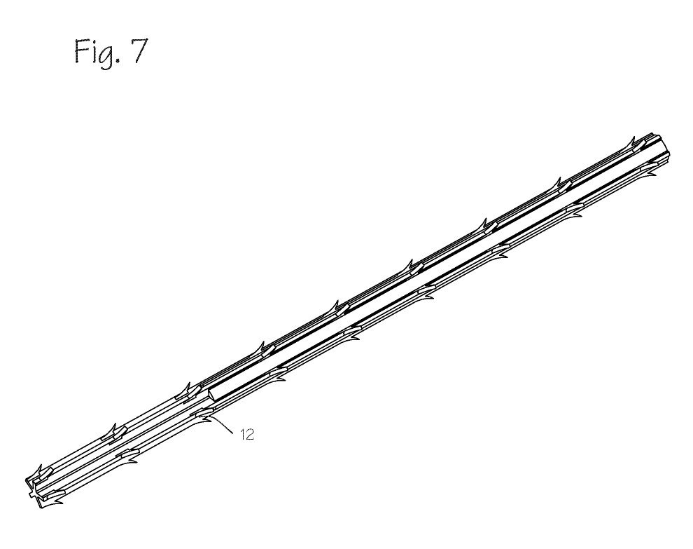

FIG. 7 illustrates an alternative stellate core suture with barbs 12. barbed suture is provided comprising a stellate core formed from a bioabsorbable polymeric material. The stellate core is a monofilament fiber having a non-circular, multi-lobed cross-sectional geometry including a plurality of longitudinally extending ribs. The stellate core may be formed from polydioxanone (PDO) or other suitable bioabsorbable polymers, and may be utilized as a standalone monofilament. A coating is disposed on at least a portion of the stellate core, including along the ribs. The coating may comprise a biodegradable binder material, such as polycaprolactone (PCL), optionally in solubilized form, and may further include one or more bioactive agents. Exemplary bioactive agents include collagen, antimicrobial agents, antibiotics, growth factors, or other therapeutic materials configured to enhance tissue interaction, reduce infection, or promote healing. A plurality of barbs are formed directly from the stellate core cutting portions of the ribs, skived, or otherwise removed to form barbs projecting outwardly from the stellate core. The barbs may be oriented at an angle relative to a longitudinal axis of the suture and may be formed in a unidirectional or bidirectional pattern. The barbs are formed directly on the coated or uncoated stellate core. The coating may be applied prior to barb formation, such that the barbs retain at least a portion of the coating, or may be applied after barb formation to coat both the stellate core and the barbed features.

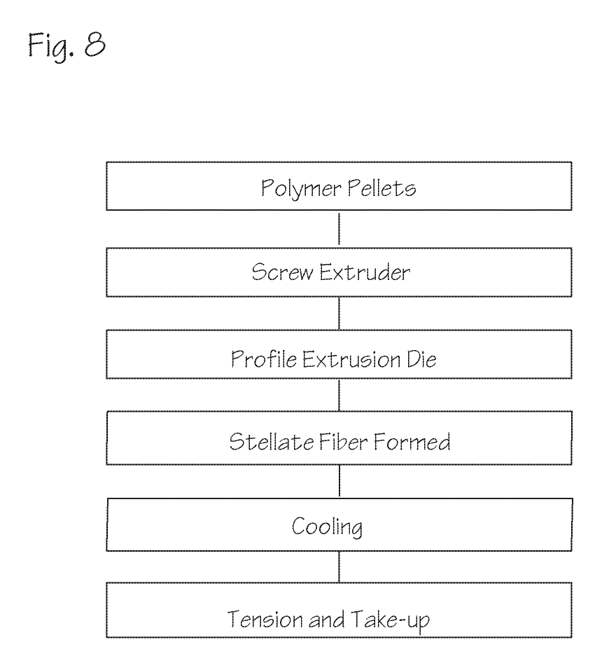

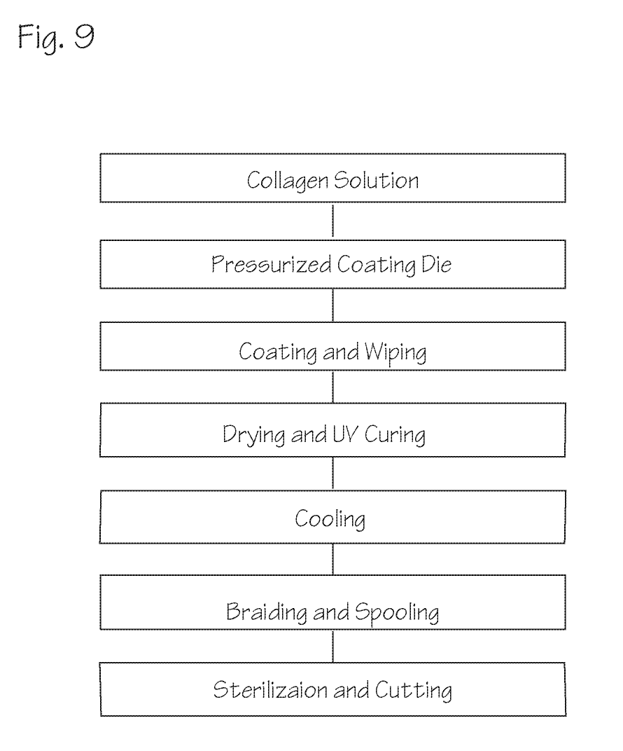

FIGS. 8 and 9 illustrate a method of manufacturing a stellate surgical suture fiber, and more particularly to a two-step process including profile extrusion of a stellate monofilament fiber in FIG. 8 and application of a collagen-based coating in FIG. 9, to enhance handling and tissue interaction. In a first step, a polymeric material suitable for surgical sutures is processed via hot melt extrusion to form a monofilament fiber having a stellate cross-section.

The extrusion system includes a screw extruder configured for heating and conveying molten polymer, a profile extrusion die configured to impart a stellate cross-sectional geometry and a die orifice having radially extending protrusions corresponding to the desired stellate shape. The polymer is introduced into the crew extruder and then heated to a molten or flowable state. The polymer is then forced through the profile extrusion die, forming a continuous stellate monofilament fiber. Upon exiting the die, the fiber undergoes a cooling process. The fiber is subjected to tension and take-up, which orients polymer chains and stabilizes the stellate geometry.

In a second step, the stellate fiber is coated with a collagen material using a pressurized coating die system. The coating apparatus includes a pressurized coating die, a material coating chamber, a coating feed system supplying coating material under controlled pressure and a coating wiper tip to regulate coating thickness. A collagen solution is prepared by dissolving collagen in a water-soluble solvent such as acetic acid that allows the collagen coating to be washed in buffered saline. The stellate fiber is first fed into the pressurized coating die and exposed to coating material delivered under pressure into the coating chamber. The center core is processed with standard thermal extrusion so that the collagen holds to the center core mechanically. The core is extruded from any suitable flexible absorbable polymer or permanent polymer or blend, of sufficient tensile strength, such as poly caprolactone, PLLA or polydioxanone or UHMWPE. The stellate shaped core may be treated with a chemical or plasma surface treatment to promote adhesion between the collagen and the polymer. The star points may have undercut features to hold the collagen to the center core. The collagen coating may be crosslinked or uncrosslinked collagen.

The extruded core is pulled through a bath of collagen slurry and through a coating die, and the collagen coating is then dried and washed. The collagen may be crosslinked using a UV light. The polymer binder may have a UV-curable crosslinking and polymerization system as well. The collagen coated star shaped core is then overbraided with polyester and/or polypropylene fibers to form the finished suture. The stellate fiber bundle may also be braided into a yarn and overcoated as a group, and then overbraided with UHMWPE and/or polyester fibers.

The collagen coating can contain fillers such as a hydrogel that cause the coating to swell and shorten the overbraid, to produce traction on the repair construct. The collagen coating may also contain osteoinductive materials such as hydroxyapatite. Optional post-processing steps can include drying or curing of the coating, crosslinking, sterilization, spooling or cutting into suture lengths.

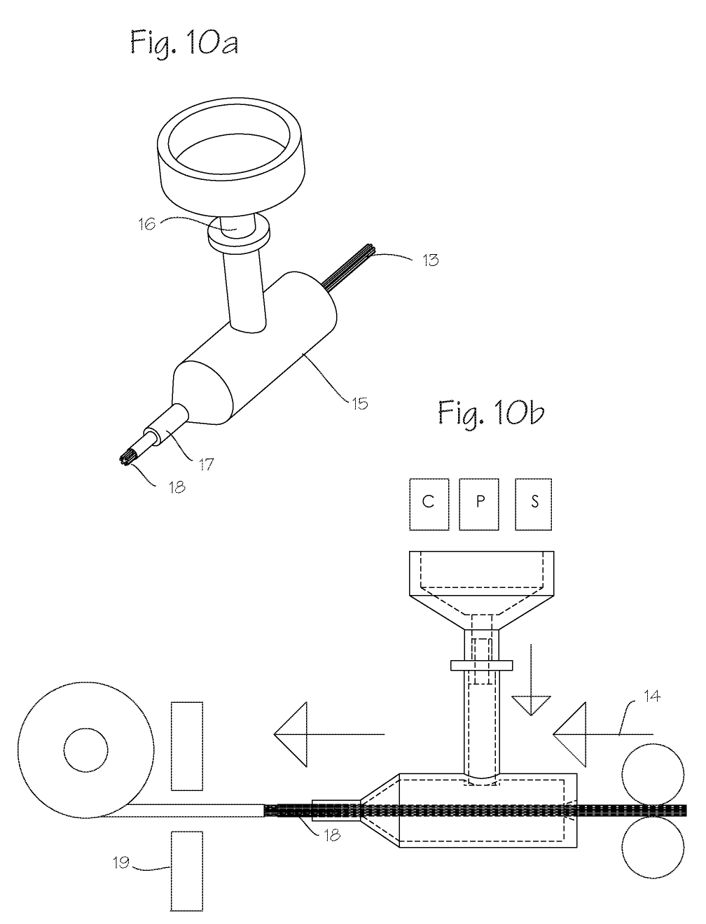

FIGS. 10a and 10b Figures illustrate another method of manufacturing a stellate core fiber using a solution-based coating process. A coating composition is prepared by dissolving a bioabsorbable polymer in a biocompatible organic solvent to form a polymer solution. Micronized collagen is added to the polymer solution to form a coating. A pre-formed stellate core fiber 13 is supplied from a feed source and advanced through the coating system. The stellate core fiber is advanced through the system using extrusion or feed rollers 14, which control line speed and coating thickness. The stellate core fiber is introduced into a pressurized coating die that defines a material coating chamber 15. The collagen-polymer coating mixture is supplied to the coating chamber under pressure from a material feed reservoir 16. As the stellate core fiber passes through the coating chamber, the coating mixture flows around and encapsulates the stellate geometry, depositing the composite coating including polymer and dispersed micronized collagen. Optionally, a coating wiper tip or die land 17 is positioned at the outlet of the coating die to remove excess coating material and control the final coating thickness. After exiting the coating die, the coated stellate core fiber enters a drying stage. The drying stage 18 may include ambient drying, heated air, infrared heating or combinations thereof. During drying, the solvent is removed, leaving a solid composite matrix comprising the polymer binder and collagen particles adhered to and surrounding the stellate core fiber 18. The dried, collagen and polymer coated stellate core fiber is collected on an uptake spool for further processing.

The coating applied to the stellate core fiber comprises a polycaprolactone (PCL)-based composite formed using a solution-based process. The coating is applied to the exterior surface and grooves of the stellate fiber. Polycaprolactone is dissolved in a biocompatible organic solvent to form a fluent polymer solution. The coating is formed by taking decellularized collagen, also known as dECM (decellularized extracellular matrix) and micronizing it. It is micronized to a dry, micron-sized powder. This can be achieved by jet milling or cryo-grinding. Suitable solvents include dichloromethane (DCM), chloroform (CL), hexafluoroisopropanol (HFIP), and combinations thereof. Micronized collagen powder is mixed into the polymer solution as a particulate filler, forming a polymer–collagen coating composition. Collagen is incorporated as a micronized particulate or suspended bioactive component within the solvent-based coating, rather than as a continuous uncrosslinked collagen phase. The coating mixture may be mixed, agitated, or sonicated to maintain uniform dispersion of collagen particles within the polymer solution. The collagen particles have a particle size in the range of about 1 to 20 microns, and more specifically, about 1 to 6 microns. The collagen remains in a particulate, micronized state within the solution and is not solubilized. Micronized collagen is then blended into the polymer–solvent solution to achieve a desired loading by volume. The collagen loading may range from approximately 5% to 80% by volume of the coating mixture. The resulting mixture forms a fluent polymer–collagen coating composition, which may be characterized as a coating “paint” suitable for application to a pre-formed stellate core fiber.

The stellate core is made of a material such as polyethylene (PE, PET, UHMWPE) that has a melt temperature of between 130°C to 140°C, that is above that of bioabsorbable polymers such as PCL. The combination of thermally extruding a coating onto the stellate core provides tensile strength to the coated fiber. The thermally extruded coating may be loaded with any heat-stable bioactive material such as heat-stabilized collagen, antibiotics or other medications. The stellate core fiber may comprise UHMWPE or another high-strength suture material and has a stellate cross-section configured to increase surface area and mechanical interlocking with the coating. The stellate fiber has a grooved cross-section having longitudinal channels or grooves.

The coating is designed to adhere to the stellate core. The binder polymer may any bioabsorbable polymer that can be dissolved with a non-toxic, biocompatible solvent. Polymers may include Polycaprolactone (PCL) epsilon PCL, polydioxanone, (PDO), Polylactic acid (PLA) Polylactic co-glycolic acid (PLGA) Polyhydroxyalkanoates, (PHA’s) PEG co-polymers, and Poly-4-hydroxybutarate (P4HB).

The collagen may be present as a suspended particulate phase within the coating. Additional active agents may be incorporated into the coating, either as suspended particulates or dissolved species. Examples of additional materials include anti-inflammatory agents, antibiotics, chemotherapeutic agents, anesthetics, growth factors, tricalcium phosphate. In addition, hydrophilic and polar materials may be added as micronized suspensions, while hydrophobic and non-polar materials may be dissolved directly into the solvent phase.

A bioabsorbable polymer binder is dissolved in a biocompatible solvent to form a fluent coating solution. In one example, Polycaprolactone (PCL) is dissolved in dichloromethane (DCM), chloroform, or Hexafluoro-2-propanol (HFIP). These solvents are highly polar, have low-viscosity, and are strong hydrogen-bonding solvents. Alternatively, solvents can include 2-methyltetrahydrofuran (2-MeTHF), Ethyl acetate (EtOAc), Methyl tert-butyl ether (MTBE), Acetic acid, Acetone or Tetrahydrofuran (THF). The polymer binder can include Polycaprolactone (PCL or ε-PCL), Polydioxanone (PDO), Polylactic acid (PLA), Polylactic-co-glycolic acid (PLGA), Polyhydroxyalkanoates (PHAs); Polyethylene glycol (PEG) copolymers; or Poly-4-hydroxybutyrate (P4HB). The types of collagen that can be used include bovine, porcine, atelocollagen or synthetic recombinant collagen.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

In the Claims:

1. A surgical suture comprising:
a core bundle comprising a plurality of elongate fibers extending longitudinally, each of the elongate fibers having a stellate cross-section defined by a plurality of radially extending arms and longitudinal channels; and
a collagen coating disposed on each elongate fiber and retained at least partially within the longitudinal channels; and
an outer braided sheath surrounding the core bundle and retaining the plurality of elongate fibers in a grouped configuration to form a unitary suture;
wherein the plurality of elongate fibers are disposed substantially parallel within the outer braided sheath.

2. The surgical suture of claim 1, wherein the collagen-containing coating is disposed at least partially within the longitudinal channels of each elongate fiber.

3. The surgical suture of claim 1, wherein the collagen-containing coating comprises:
micronized collagen particles dispersed within a polymer binder.

4. The surgical suture of claim 3, wherein the collagen particles have a size between about 1 micron and about 5 microns, and the collagen is present in an amount between about 5% and about 80% by volume of the coating.

5. The surgical suture of claim 3, wherein the polymer binder comprises polycaprolactone (PCL).

6. The surgical suture of claim 5, wherein the plurality of elongate fibers comprises between 20 and 30 fibers.

7. The surgical suture of claim 1, wherein the suture has a nominal diameter between about 500 microns and about 600 microns.

8. The surgical suture of claim 1, wherein the elongate fibers comprise ultra-high molecular weight polyethylene (UHMWPE), polyester, or a combination thereof.

9. The surgical suture of claim 1, wherein the outer braided sheath comprises UHMWPE, polyester, or a combination thereof.

10. A surgical suture comprising:
a plurality of elongate fibers interlaced with one another to form a braided structure extending along a longitudinal axis, wherein each of the elongate fibers has a stellate cross-section defined by a plurality of radially extending arms and longitudinal channels; and
a collagen-containing coating on all of the fibers, wherein the collagen-containing coating is disposed at least partially within the longitudinal channels.

11. The surgical suture of claim 10, wherein the collagen-containing coating comprises micronized collagen particles dispersed within a polymer binder.

12. The surgical suture of claim 10, wherein:
the collagen particles have a size between about 1 micron and about 5 microns; and the collagen is present in an amount between about 5% and about 80% by volume.

13. The surgical suture of claim 11, wherein the polymer binder comprises polycaprolactone (PCL).

14. The surgical suture of claim 10, wherein the plurality of elongate fibers comprises between 20 and 30 fibers.

15. The surgical suture of claim 10, wherein the suture has a nominal diameter between about 500 microns and about 600 microns.

16. The surgical suture of claim 10, wherein the elongate fibers comprise ultra-high molecular weight polyethylene (UHMWPE), polyester, or an absorbable polymer.

17. The surgical suture of claim 10, wherein the stellate cross-sectional geometry increases surface area relative to cylindrical fibers of equal diameter.
